(19) United States
(12) Patent Application Publication
(10) Pub. No.: US 2010/0062430 A1
(43) Pub. Date: Mar. 11, 2010

(54) METHOD AND KIT FOR MOLECULAR CHROMOSOMAL QUANTIFICATION

(75) Inventors: Dan Hauzenberger, Saltsjo-Boo (SE); Ulf Klangby, Sollentuna (SE); Anders Hedrum, Alta (SE)

Correspondence Address: MORRISON & FOERSTER LLP, 425 MARKET STREET, SAN FRANCISCO, CA, 94105-2482, US

(73) Assignee: Vytal Diagnostics AB, Stockholm (SE)

(21) Appl. No.: 12/298,127

(22) PCT Filed: Apr. 26, 2007
(86) PCT No.: PCT/SE07/50276

§ 371 Date: Oct. 27, 2009

Related U.S. Application Data
(60) Provisional application No. 60/795,159, filed on Apr. 27, 2006.

(51) Int. Cl. C12Q 1/68; C07H 21/00

(52) U.S. Cl. 435/6; 536/24.33

(57) ABSTRACT

Diagnosis of chromosomal abnormalities or genetic disorders is performed using at least two marker sequences, wherein one marker sequence is a sequence known to be present on the chromosome or in the gene of interest, another marker sequence is a sequence known to be present on an autosomal chromosome, and the marker sequences are partially homologous. A kit for performing this diagnosis is also claimed.

### FIELD OF THE INVENTION

The present invention relates to a novel method for use in the detection or diagnosis of chromosomal abnormalities or genetic disorders. The invention in particular relates to a method for relative molecular quantification of chromosomes enabling the detection of both chromosomal aneuploidies as well as quantitative and qualitative gene aberrations. The invention further relates to a diagnostic kit and a screening method.

### BACKGROUND

It is well known that the risk of foetal chromosomal anomalies (e.g. Down's syndrome) increases with maternal age. Prenatal diagnostics answers the need to detect early in pregnancy a number of chromosomal anomalies. The prenatal diagnosis of chromosomal anomalies has become widely available for pregnancies at risk in the last three decades. The risk increases with age and a markedly increased risk is seen in mothers aged 35 or more. Chromosomal anomalies frequently involve trisomy 21 (Down's syndrome), but also trisomies 13 and 18 and sex chromosome defects are frequently observed in children born by mothers in this age group. At the age of 20 the risk of trisomy 21 is approximately 1/2000, 1/1200 at 25, 1/900 at 30, 1/400 at 35, 1/100 at 40 and 1/40 at 45 years of age.

Karyotyping is the most frequently used test method on material obtained by invasive-techniques such as CVS and amniocentesis. Karyotyping detects a range of numerical and structural chromosome abnormalities in addition to the common autosomal trisomies 13 (Patau's syndrome), 18 (Edwards' syndrome) and 21 (Down's syndrome) as well as sex chromosome abnormalities e.g. X0 (Turner's syndrome) and XXY (Klinefelter's syndrome). However, since amniotic fluid and chorionic villus cells are cultured before analysis, delays of up to 14 days or longer can occur before the results are available. Molecular methods based on Polymerase Chain Reaction (PCR) and DNA probe hybridisation have therefore been developed

(Non Patent Citation 0001: HULTEN, M A. Rapid and simple prenatal diagnosis of common chromosome disorders: advantages and disadvantages of the molecular methods FISH and QF-PCR. Reproduction, 2003 vol. 126, no. 3, p. 279-97.

and

Non Patent Citation 0002: NICOLINI, U. The introduction of QF-PCR in prenatal diagnosis of fetal aneuploidies: time for reconsideration. Human Reproduction Update, 2004 vol. 10, no. 6, p. 541-548.)
These techniques are faster than karyotyping and can be used for the common autosomal and sex chromosome aneuploidies mentioned above. Among the techniques currently used are FISH (fluorescent-in-situ-hybridization) of non-cultured cells and QF-PCR (Quantitative Fluorescent PCR). These techniques generate results within 48 hrs but may have limitations in detecting chromosomal aneuploidies within mosaicism
(Non Patent Citation 0003: CAINE, A. Prenatal detection of Down's syndrome by rapid aneuploidy testing for chromosomes 13, 18, and 21 by FISH or PCR without a full karyotype: a cytogenetic risk assessment. Lancet, 2005 vol. 366, no. 9480, p. 123-8.)
and/or in samples with maternal cell contamination (MCC). However, the fact that these test generate results within 24-48 hrs enabling early decisions on pregnancy management for abnormal foetuses has led to a technique shift for prenatal screening in many laboratories.

QF-PCR is based on a technology where chromosome-specific, repeated DNA sequences (known as short tandem repeats (STRs) are amplified by PCR. The use of fluorescently labelled primers allows visualisation and quantification of the fluorecently labeled PCR products. Quantification may be performed by calculating the ratio of the specific peak areas of the respective repeat lengths using an automated DNA sequencer. STRs vary in length between subjects, depending on the number of times the tri-, tetra- or penta-nucleotides are repeated. DNA amplified from normal subjects who are heterozygous (have alleles of different lengths) is expected to show two peaks with the same peak areas. DNA amplified from subjects who are trisomic will exhibit either an extra peak (being triallelic) with the same area, or only two peaks (being diallelic), one of them twice as large as the other

(Non Patent Citation 0004: NICOLINI, U. The introduction of QF-PCR in prenatal diagnosis of fetal aneuploidies: time for reconsideration. Human Reproduction Update, 2004 vol. 10, no. 6, p. 541-548.)
Subjects who are homozygous (have alleles of same length) or monsosomic will display only one peak.

The inability of the QF-PCR technique to distinguish subjects who are homozygous or monosomic is a major shortcoming when testing for sex chromosome abnormalities. When STRs specific for chromosome X are used, some samples from normal females (46,XX) may show homozygous QF-PCR patterns, indistinguishable from those produced by samples with a single X, as in Turner's syndrome (45,X). Incorporating additional X-chromosome STR markers into the analysis will reduce but not eliminate the likelihood of homozygosity

(Non Patent Citation 0005: DONAGHUE, C. Development and targeted application of a rapid QF-PCR test for sex chromosome imbalance. Prenat Diagn, 2003 vol. 23, no. 3, p. 201-10.)

Prenatal diagnosis of Turner's syndrome by determining the abscence of a methylated copy of the FMR-1 gene on the X-chromosome has been described as potentially useful method for detection of Turner's syndrome

(Non Patent Citation 0006: PENA, S D J. Fetal diagnosis of monosomy X (Turner's syndrome) with methylation-specific PCR. Prenatal Diagnosis, 2003 vol. 23, p. 769-770.)

Genotyping the X-Y homologous amelogenin (AMELX and AMELY) gene segments for gender identification is widely used for DNA profiling in prenatal diagnoses. Regions on this gene are sufficiently conserved and may be amplified, using identical primers, for simultaneous detection of the AMELX and AMELY alleles in gender identification procedures. When amplification of AMELX and AMELY is used in the QF-PCR procedure it may also be helpful in providing a quantitative relationship between chromosomes X and Y. However, no quantitative information for the X-chromosome will be obtained in females as the AMELY gene is not present.

Relative quantification of the X-chromosome by co-amplification of a X-chromosome STR marker (HPRT) versus a chromosome 21 (D21S1411) STR marker using separate primer pairs has been suggested as an alternative strategy for QF-PCR to diagnose monosomy X. The method does not require a heterozygous pattern to be obtained for the STR markers but assumes an identical amplification efficiency of the two different primer pairs amplifying two different STR markers

(Non Patent Citation 0007: CIRIGLIANO, V. X chromosome dosage by quantitative fluorescent PCR and rapid prenatal diagnosis of sex chromosome aneuploidies. Molecular Human reproduction, 2002 vol. 8, no. 11, p. 1042-1045.)

Consequently there remains a need for an improved method setting aside the shortcomings and disadvantages associated with known methods.

### SUMMARY

In developing a method for quantification of chromosomes and genes in a sample taken from a mammal, the present inventors have surprisingly found that the above shortcomings and disadvantages can be set aside by choosing at least two marker sequences, wherein one marker sequence is a sequence known to be present on the chromosome or in the gene of interest, another marker sequence is a sequence known to be present on an autosomal chromosome, and the marker sequences are partially homologous. According to this method, the sample is amplified using substantially homologous PCR primer pairs hybridising to the marker sequences known to be present on the chromosomes or genes of interest, and amplified DNA fragments are detected.

The invention is defined in the attached claims, incorporated herein by reference.

### SHORT DESCRIPTION OF THE DRAWINGS

The invention will be described in closer detail in the following description, non-limiting examples and claims, with reference to the attached drawings in which

FIG. 1 illustrates an analysis according to the background technology where, using QF-PCR and chromosome specific STRs, it is not possible to distinguish between subjects who are homozygous or monosomic. In this test, normal heterozygous subjects will display two peaks with the same peak area. DNA amplified from trisomic subjects will exhibit an extra peak (being triallelic), or only two peaks (being diallelic), whereas subjects who are homozygous or monosomic will display only one peak.

FIG. 2 illustrates an embodiment of the invention where one primer pair is used, specific for a marker sequence on a sex chromosome, as well as for a marker sequence on an autosomal chromosome, the two marker sequences being at least partially homologous and of different length.

FIG. 3a shows a normal Male (46,XY) chromatogram as displayed by the presence of AMELX and AMELY in a 1:1 ratio. A single allele of the X-specific DXS1187 marker and a 2:1 area ratio of chromosome 7 (BRAF7) to chromosome X (BRAFX).

FIG. 3b shows a normal female (46,XX) chromatogram as displayed by the presence of AMELX and absence of AMELY. A two allele pattern in a 1:1 ratio of the X-specific DXS1187 marker and a 1:1 area ratio of chromosome 7 (BRAF7) to chromosome X (BRAFX).

FIG. 3c shows a Turner Syndrom, X0 (45,X), female chromatogram as displayed by the presence of AMELX and absence of AMELY. A one allele pattern of the X-specific DXS1187 marker and a 2:1 area ratio of chromosome 7 (BRAF7) to chromosome X (BRAFX).

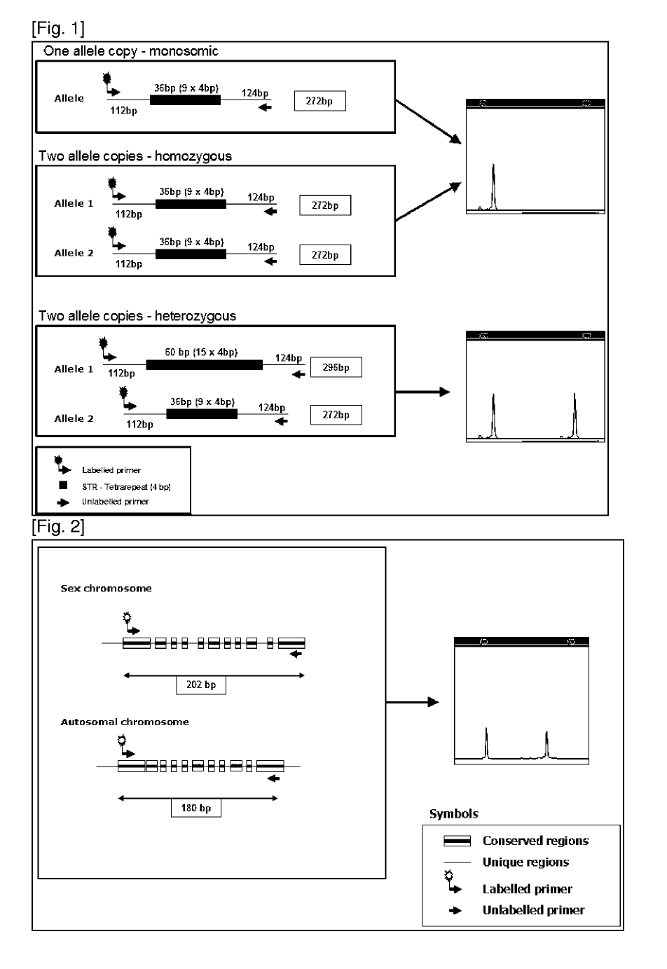

### DETAILED DESCRIPTION

Before the present device and method is described, it is to be understood that this invention is not limited to the particular configurations, method steps, and materials disclosed herein as such configurations, steps and materials may vary somewhat. It is also to be understood that the terminology employed herein is used for the purpose of describing particular embodiments only and is not intended to be limiting since the scope of the present invention will be limited only by the appended claims and equivalents thereof.

It must also be noted that, as used in this specification and the appended claims, the singular forms “a”, “an”, and “the” include plural referents unless the context clearly dictates otherwise.

The term “about” when used in the context of numeric values denotes an interval of accuracy, familiar and acceptable to a person skilled in the relevant art. Said interval can be ±10% or preferably ±5%.

In describing and claiming the present invention, the following terminology will be used in accordance with the definitions set out herein.

The term “sample” here means a volume of a liquid, solution, biopsy or cell suspension, taken from an organism, such as a mammal, preferably a human. The sample may be subjected to qualitative or quantitative determination according to the invention as such, or after suitable pre-treatment, such as homogenisation, sonication, filtering, sedimentation, centrifugation, etc.

Typical samples in the context of the present invention are body fluids such as blood, plasma, serum, amniotic fluid, lymph, urine, saliva, semen, gastric fluid, sputum, tears as well as tissue samples such as Chorinic Villus Samples (CVS) etc.

Further in the context of this invention, the terms “hybridisation” and “hybridisable” refer to hydrogen bonding, which may be Watson-Crick, Hoogsteen or reversed Hoogsteen hydrogen bonding, between complementary nucleoside or nucleotide bases. For example, adenine and thymine are complementary nucleobases, which pair through the formation of hydrogen bonds.

“Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “hybridisable” and “complementary” are terms which are used to indicate a sufficient degree of complementarity or precise pairing such that stable and specific binding occurs between the oligonucleotide and the DNA or RNA target.

The expression “partially homologous” or “partially identical” refers to a relationship or degree of identity between two sequences, here preferably two marker sequences, each having a part of its sequence homologous to a part of the other. The homology between said parts is defined as at least about 75%, preferably at least about 80%, and most preferably at least about 90 or even more preferably about 95% of the nucleotides match over said parts. Partially homologous therefore allows a low homology over the entire length of the sequences, as long as two defined parts exhibit a high homology of at least about 75% or higher.

The expression “substantially homologous” or “substantially identical” refers to a relationship or degree of identity between two sequences, here preferably a primer and a marker sequence, and requires that at least about 85%, preferably at least about 90%, and most preferably at least about 95 or even more preferably about 100% of the nucleotides match over the defined length of the DNA sequences. Sequences that are substantially homologous can be identified by comparing the sequences using standard software available in sequence data banks, or in a Southern hybridisation experiment under, for example, stringent conditions as defined for that particular system. Defining appropriate hybridisation conditions is within the skill of the art and guidance can be found in literature, e.g. in

### Non Patent Citation 0008: SAMBROOK, J. Molecular Cloning: A Laboratory Manual. 3: Cold Spring Harbor Laboratory Press, 2001. ISBN 0879695773.

“Homology” or degree of identity can also be determined using applicable software, known and available to persons skilled in the art. Examples of such software include, but are not limited to ClustaIW (available for download at the website http://www.ebi.ac.uk/clustalw/) and NCBI BlastAlign (available at the website http://www.bio.ic.ac.uk/research/belshaw/BlastAlign.tar).

The inventors here present a technique using sex chromosome-specific DNA sequences or marker sequences that are partially homologous to sequences specific for a second autosomal chromosome. These DNA sequences can be utilized for relative and absolute gene and/or chromosome quantification using methods and diagnostic kits as defined in the attached claims, hereby incorporated in their entirety. The utilization of the here described technique enables the quantification of genes and or chromosomes irrespectively of whether individuals are homozygous or carrying true chromosomal aneuploidies. This is a significant advantage compared to previous techniques which are associated with the risk of misdiagnosing homozygous individuals. Most important is the ability to quantify both genes and chromosomes using molecular biology techniques. This makes available completely novel possibilities to detect homozygous and heterozygous monogeneic and multigeneic disorders as well as chromosomal aneuploidies using relative quantitative and true quantitative molecular biology techniques.

The invention comprises the design or selection of at least two oligonucleotide sequences that are substantially homologous to genomic sequences present on at least two chromosomes of interest. The oligonucleotide sequences are further designed or chosen so that the genomic sequences between the oligonucleotide sequences on the chromosome of interest are partially homologous, thus resulting in amplification of fragments of separate sizes and/or separate nucleotide sequences. These amplified fragments may be directly quantified using true quantitative molecular techniques such as real-time PCR where the two genomic sequences are distinguished by their partially non-homologous nucleotide sequences. These amplified fragments can also be quantified using relative molecular quantification techniques as for instance QF-PCR where the amplified nucleotide fragments are distinguished by the separate sizes and/or nucleotide sequences of the amplified fragments as determined in a post-amplification detection step.

Both quantifying techniques, QF-PCR and real-time PCR, rely on comparable PCR amplification efficiency of all oligonucleotides included in the reaction. PCR, an abbreviation for Polymerase Chain Reaction, is a technique to exponentially amplify a small quantity of a specific nucleotide sequence in the presence of template sequence, two oligonucleotide primers that hybridize to opposite strands and flank the region of interest in the target DNA, and a thermostable DNA polymerase. The reaction is subjected to different temperatures in cycles involving template denaturation, primer annealing, and the extension of the annealed primers by DNA polymerase until enough copies are made for further analysis. The performing of PCR analyses per se is considered well known to a skilled person, having access to reagents, apparatuses and protocols from many different suppliers.

According to one embodiment of the invention, the marker sequences are partially homologous and of different length, the length difference being sufficient to distinguish the amplification products during detection.

According to another embodiment of the invention, the marker sequences are partially homologous but being sufficiently different in sequence to distinguish the amplification products by the sequence in between the PCR primers.

According to one embodiment, the method according to the invention is used for detection and/or diagnosis of partial or complete chromosomal aneuploidies.

According to another embodiment, the method according to the invention is used for detection and/or diagnosis of partial or complete chromosomal monosomies. One example of a chromosomal monosomy is Turner's syndrome (X0).

According to yet another embodiment, the method according to the invention is used for detection and/or diagnosis of genetic disorders.

In an example used to demonstrate the utility of the invention, the pair of marker sequences were the BRAF-gene on chromosome 7 (BRAF7), and the BRAF2-gene on chromosome X (BRAFX). In that example, the marker sequences were amplified using the primers shown in the examples as SEQ ID NO. 1 and SEQ ID NO. 2 (See below).

Additional PCR primer sequences and suggested combinations for simultaneous amplification of BRAF7 and BRAFX include, but are not limited to the following:

(SEQ ID NO 1)

| | |
|---|---|
| Forward primer: | GGGGAACGGAACTGATTTTT |

(SEQ ID NO 2)

| | |
|---|---|
| Reverse primer: | TGTTGGGCAGGAAGACTCTAA |

(SEQ ID NO 3)

| | |
|---|---|
| Reverse primer: | TTGTTGGGCAGGAAGACTCTA |

(SEQ ID NO 4)

| | |
|---|---|
| Reverse primer: | TGTTGGGCAGGAAGACTCTA |

(SEQ ID NO 5)

| | |
|---|---|
| Reverse primer: | GTGGTGACTTGGGGTTGCT |

(SEQ ID NO 6)

| | |
|---|---|
| Forward primer: | CTGGGGAACGGAACTGATT |

(SEQ ID NO 7)

| | |
|---|---|
| Reverse primer: | TGTTGGGCAGGAAGACTCTAA |

(SEQ ID NO 8)

| | |
|---|---|
| Reverse primer: | TTGTTGGGCAGGAAGACTCTA |

(SEQ ID NO 9)

| | |
|---|---|
| Reverse primer | TGGTGACTTGGGGTTGCT |

(SEQ ID NO 10)

| | |
|---|---|
| Forward primer: | CTGGGGAACGGAACTGATTT |

(SEQ ID NO 11)

| | |
|---|---|
| Reverse primer: | TTGTTGGGCAGGAAGACTC |

(SEQ ID NO 12)

| | |
|---|---|
| Forward primer: | TGGGGAACGGAACTGATTT |

(SEQ ID NO 13)

| | |
|---|---|
| Forward primer: | AACCCCAAGTCACCACAAAA |

(SEQ ID NO 14)

| | |
|---|---|
| Reverse primer: | TTGTGGTGACTTGGGGTTG |

(SEQ ID NO 15)

| | |
|---|---|
| Reverse primer: | TTTGTGGTGACTTGGGGTTG |

(SEQ ID NO 16)

| | |
|---|---|
| Forward primer: | CAACCCCAAGTCACCACAA |

(SEQ ID NO 17)

| | |
|---|---|
| Reverse primer: | GTGGTGACTTGGGGTTGC |

The above sequences can be used in different combinations, for example as shown below:
1. SEQ ID NO 1 and SEQ ID NO 2. Expected product size (X-chromosome): 203 bp
2. SEQ ID NO 1 and SEQ ID NO 3. Expected product size (X-chromosome): 204 bp
3. SEQ ID NO 13 and SEQ ID NO 3. Expected product size (X-chromosome): 50 bp
4. SEQ ID NO 1 and SEQ ID NO 4. Expected product size (X-chromosome): 203 bp
5. SEQ ID NO 1 and SEQ ID NO 14. Expected product size (X-chromosome): 172 bp
6. SEQ ID NO 1 and SEQ ID NO 15. Expected product size (X-chromosome): 173 bp
7. SEQ ID NO 1 and SEQ ID NO 5. Expected product size (X-chromosome): 170 bp
8. SEQ ID NO 6 and SEQ ID NO 7. Expected product size (X-chromosome): 205 bp
9. SEQ ID NO 6 and SEQ ID NO 8. Expected product size (X-chromosome): 206 bp
10. SEQ ID NO 1 and SEQ ID NO 17. Expected product size (X-chromosome): 170 bp
11. SEQ ID NO 1 and SEQ ID NO 9. Expected product size (X-chromosome): 169 bp
12. SEQ ID NO 10 and SEQ ID NO 7. Expected product size (X-chromosome): 205 bp
13. SEQ ID NO 10 and SEQ ID NO 8. Expected product size (X-chromosome): 206 bp
14. SEQ ID NO 1 and SEQ ID NO 11. Expected product size (X-chromosome): 204 bp
15. SEQ ID NO 12 and SEQ ID NO 7. Expected product size (X-chromosome): 204 bp
16. SEQ ID NO 12 and SEQ ID NO 8. Expected product size (X-chromosome): 205 bp
17. SEQ ID NO 13 and SEQ ID NO 11. Expected product size (X-chromosome): 50 bp
18. SEQ ID NO 6 and SEQ ID NO 14. Expected product size (X-chromosome): 174 bp
19. SEQ ID NO 16 and SEQ ID NO 7. Expected product size (X-chromosome): 50 bp
20. SEQ ID NO 16 and SEQ ID NO 3. Expected product size (X-chromosome): 51 bp

The primers can be distinguished not only by differences in length, but may also contain suitable marker functionalities, such as fluorescent markers or the like, well known to a person skilled in the art.

An embodiment of the present invention further makes available a diagnostic kit including reagents for performing the method defined above.

Another embodiment of the invention also makes available a screening method, characterized in that the method defined above is used.

### EXAMPLES

### 1. Design and Amplification of Nucleotide Sequences Used to Distinguish Sex Chromosome Aneuploidies Using QF-PCR

In order to verify the ability of the invention to quantify the number of chromosomes in an unknown sample the following experimental conditions were used. PCR primers with sequences homologous to sequences present in the BRAF-gene on chromosome 7 (NCBI Acc. No NC_{—}000007) as well as in the BRAF2-gene on chromosome X (NCBI Acc. No NC_{—}000023) were designed. The PCR primer sequences used for amplification were as follows:

(SEQ ID NO 1)

| | |
|---|---|
| Forward primer: | 5′-GGGGAACGGAACTGATTTTT-3′ |

(SEQ ID NO 2)

| | |
|---|---|
| Reverse primer: | 5′-HEX-TGTTGGGCAGGAAGACTCTAA-3′. |

These PCR primer sequences were used to amplify a fragment of approximately 182 bp from chromosome 7 and a fragment of approximately 203 bp from chromosome X, respectively. PCR primers for the following genetic markers were always included in the multiplex PCR reaction: AMEL, DXS1187, SRY, DXS981 and XHPRT (see table 1 for details). A skilled person will be able to identify additional markers using routine experimentation in silico.

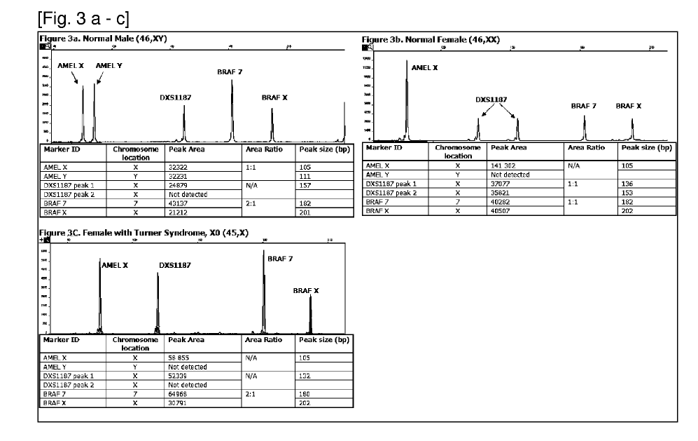

The DNA purification and PCR reactions were set up and performed as follows: Cells were obtained by amniocentesis or by cell culture. Cells were enriched and washed using standard centrifugation and PBS. Following enrichment and washing, DNA was extracted and purified using QIAamp DNA Blood Kit (Qiagen, Germany) and InstaGene Matrix (Bio-Rad Laboratories, UK). Purified nucleic acids were subsequently subjected to PCR amplification as described below. In brief, 5 μl of DNA (1-10 ng/μl) was added to the PCR reaction containing Taq-polymerase (2U/reaction), PCR primers (0.02 μM forward and reverse primers, respectively) and a buffer containing 50 mM KCl, 15 mM Tris-HCl pH 8.0. The sample was subjected to PCR amplification using Thermal Cycler GeneAmp® PCR System 9700 using the following conditions; 95° C. 15 min; 94° C. 30 sec; 58° C. 90 sec; 72° C. 90 sec for 26 cycles, 72° C. 30 min and 4° C. forever. 3 μl of the denatured and amplified sample was subsequently analysed on an ABI PRISM® Genetic Analyzer as described in the addendum; ABI PRISM® Genetic Analyzers User Manual. Gene-Scan-500 ROX was used as internal size standard. The results of such amplified and separated PCR fragments are shown in FIGS. 3a-c.

TABLE 1
Examples of genetic markers

| Marker ID | Location | Type | NCBI Acc. No |
|---|---|---|---|
| BRAF | Chr X; | Non variable | X: NC_000007 |
| | Chr 7 | | Y: NC_000023 |
| AMEL | Chr X; | Non variable | X: NC_000023 |
| | Chr Y | | Y: NC_000024 |
| DXS1187 | Chr X | STR | NC_000023 |
| DXS981 | Chr X | STR | NC_000023 |
| SRY | Chr Y | Non variable | NC_000024 |
| XHPRT | Chr X | STR | NC_000023 |

A total of 303 clinical samples were analysed, whereof 94 blood samples, 204 amniotic fluid samples and 5 cell lines. The samples were analysed using the experimental conditions and the diagnostic kit described below. In addition, the amniotic fluid samples were also analyzed in parallel using karyotyping.

58 of the blood samples were determined to be male with all X-chromosomal STR markers homozygous and the BRAF (7:X) marker displaying an expected 2:1 ratio. 36 of the blood samples were determined female with at least one X-chromosomal STR markers heterozygous and with the expected 1:1 BRAF (7:X) ratio. One female sample was homozygous for all X-chromosomal STR markers tested but displayed a normal female 1:1 BRAF (7:X) ratio. Results from all tested blood samples are summarised in Tables 2a (female blood samples) and 2b (male blood samples).

A total of 102 amniotic fluid samples and cell lines were independently determined as females by QF-PCR and karyotyping (table 3a and FIG. 3b). Moreover, two of the female samples were independently determined as 45,X (table 3a and FIG. 3c) by QF-PCR and karyotyping. All X-chromosomal STR markers were homozygous and the BRAF (7:X) marker showed an abnormal female 2:1 ratio in QF-PCR for both 45,X samples. A total of 107 amniotic fluid samples and cell lines were independently determined as male by QF-PCR and karyotyping (Table 3b and FIG. 3a). Moreover, two of the male samples were determined as 47,XXY by QF-PCR and karyotyping. At least one X-chromosomal STR marker was heterozygous and the BRAF (7:X) marker showed an abnormal male 1:1 ratio in QF-PCR for both 47,XXY samples.

Results from QF-PCR in 94 Blood Samples

TABLE 2a
Female blood samples

7:X ratio

| | 2:1 | 1:1 |
|---|---|---|
| Total | 0 | 36 |
| All X markers | 0 | 1 |
| Homozygous | | |
| At least one X marker heterozygous | 0 | 35 |
| Y chromosome detected | 0 | 0 |

TABLE 2b
Male blood samples

7:X ratio

| | 2:1 | 1:1 |
|---|---|---|
| Total | 58 | 0 |
| All tested X markers | 58 | 0 |
| Homozygous | | |
| At least one X marker heterozygous | 0 | 0 |
| Y chromosome detected | 58 | 0 |

Results from QF-PCR in 209 Amniotic Fluid and Cell Line Samples

TABLE 3a
Female amniotic fluid and cell line samples

7:X ratio

| | 2:1 | 1:1 |
|---|---|---|
| Total | 2* | 100** |
| All tested X markers | 2* | 0 |
| Homozygous | | |
| At least one X marker heterozygous | 0 | 100** |
| Y chromosome detected | 0 | 0 |

*Karyotyping results: 45, X
**Karyotyping results: 46, XX

TABLE 3b
Male amniotic fluid and cell line samples

7:X ratio

| | 2:1 | 1:1 |
|---|---|---|
| Total | 105* | 2** |
| All tested X markers | 105* | 0 |
| Homozygous | | |
| At least one X marker heterozygous | 0 | 2** |
| Y chromosome detected | 105* | 2** |

*Karyotyping results: 46, XY
**Karyotyping results: 47, XXY

### 2. Diagnostic Kit

A diagnostic kit (Devyser Complete™, Devyser AB, Stockholm, Sweden) was used for fetal diagnosis of Turner's syndrome in amniotic fluid obtained from pregnant women.

The diagnostic kit included the following reagents: a PCR reagent mix (Mix2), containing primer sets for detection of BRAF, AMEL, DXS1187, SRY and XHPRT in a buffered Mg 2+ solution, and a PCR activator mix (PCR activator), containing DNA polymerase in a buffered solution. In addition the kit included a second PCR reagent mix (Mix1) for analysis of STR markers present on chromosomes 13, 18 and 21.

The diagnostic kit was used according to the inventive method. Briefly, the DNA purification was set up and performed as follows: Amniotic fluid was obtained by amniocentesis. Amniocytes were enriched from the amniotic fluid and washed using standard centrifugation and PBS. Following enrichment and washing, DNA was extracted and purified using QIAamp DNA Blood Kit (Qiagen, Germany) or InstaGene Matrix (Bio-Rad Laboratories, UK).

The PCR reactions were set up and performed as follows. PCR reaction mixes were prepared by addition of 10 μL PCR Activator to each Mix1 and Mix2, respectively. 20 μL of each of the reaction master mixes were subsequently distributed to PCR vials and 5 μL purified nucleic acid was added to each of Mix1 and Mix2. Positive (Normal male genomic DNA) and Non-template controls were included in each run.

The samples were subjected to PCR amplification using a Thermal Cycler using the following conditions; 95° C. 15 min; 94° C. 30 sec; 58° C. 90 sec; 72° C. 90 sec for 26 cycles, 72° C. 30 min and 4° C. until termination of the run. 1.5 μl of the amplified sample was mixed with 15 μl deionised formamide, containing a suitable size standard, and subsequently analysed on an ABI PRISM® 3130 Genetic Analyzer as described in the instructions for use provided with the diagnostic kit.

### 3. Quantitative and Qualitative Molecular Analysis of Genetic Status in DNA

Although the above mentioned method to distinguish chromosomal aneuploidies in this application has been demonstrated using QF-PCR, the detection of such gene and/or chromosomal aberration using nucleic acids can also be performed using other adequate molecular techniques such as: end-point PCR detection (including for example QF-PCR, PCR combined with detection using gel analysis, DNA arrays or MALDI-TOF etc), real-time detection PCR (including for example Dual-labelled probes, self-probing amplicons, intercalating dyes etc). These techniques are well known to persons skilled in the art, and the apparatuses, reagents and kits are commercially available from several suppliers. Given the teaching in the present description, examples and claims, a skilled person can adapt existing protocols and perform the invention.

Although the invention has been described with regard to its preferred embodiments, which constitute the best mode presently known to the inventors, it should be understood that various changes and modifications as would be obvious to one having the ordinary skill in this art may be made without departing from the scope of the invention as set forth in the claims appended hereto.

### REFERENCES

HULTEN, M A, et al. Rapid and simple prenatal diagnosis of common chromosome disorders: advantages and disadvantages of the molecular methods FISH and QF-PCR. Reproduction. 2003, vol. 126, no. 3, p. 279-97.
NICOLINI, U, et al. The introduction of QF-PCR in prenatal diagnosis of fetal aneuploidies: time for reconsideration. Human Reproduction Update. 2004, vol. 10, no. 6, p. 541-548.
CAINE, A, et al. Prenatal detection of Down's syndrome by rapid aneuploidy testing for chromosomes 13, 18, and 21 by FISH or PCR without a full karyotype: a cytogenetic risk assessment. Lancet. 2005, vol. 366, no. 9480, p. 123-8.
NICOLINI, U, et al. The introduction of QF-PCR in prenatal diagnosis of fetal aneuploidies: time for reconsideration. Human Reproduction Update. 2004, vol. 10, no. 6, p. 541-548.
DONAGHUE, C, et al. Development and targeted application of a rapid QF-PCR test for sex chromosome imbalance. Prenat Diagn. 2003, vol. 23, no. 3, p. 201-10.
PENA, S D J. Fetal diagnosis of monosomy X (Turner's syndrome) with methylation-specific PCR. Prenatal Diagnosis. 2003, vol. 23, p. 769-770.
CIRIGLIANO, V. X chromosome dosage by quantitative fluorescent PCR and rapid prenatal diagnosis of sex chromosome aneuploidies. Molecular Human reproduction. 2002, vol. 8, no. 11, p. 1042-1045.
SAMBROOK, J, et al. Molecular Cloning: A Laboratory Manual. 3rd edition. Cold Spring Harbor Laboratory Press, 2001. ISBN 0879695773.

1. A method for quantification of chromosomes and genes in a sample taken from a mammal, in the diagnosis of chromosomal abnormalities or genetic disorders, the method comprising:
a) choosing at least two genetic markers, wherein
at least one genetic marker comprises a pair of marker sequences wherein one marker sequence is a sequence known to be present on the X-chromosome and another marker sequence is a sequence known to be present on an autosomal chromosome, and
at least one other genetic marker comprises sex chromosome marker sequence(s),
b) amplifying the sample using at least two primers for each genetic marker and the primers being substantially homologous to and hybridise to the marker sequences known to be present on the chromosomes or genes of interest, and wherein the marker sequences within the pair of marker sequences are partially homologous and of different length, the length difference being sufficient to distinguish the amplification products during detection,
c) detecting the amplified fragments; and
d) determining the ratio of said amplification products from the pair of marker sequences, wherein a ratio of the amplification products from the pair of marker sequences known to be present on the X-chromosome and on an autosomal chromosome which is not 1:1 in a sample determined by the second genetic marker to be obtained from a female is indicative of an X-chromosomal disorder, and wherein a ratio which is not 2:1 in a sample determined by the second genetic marker to be obtained from a male is indicative of an X-chromosomal disorder.

2. The method according to claim 1, wherein at least three genetic markers are chosen and at least one genetic marker of said at least three genetic markers comprises a STR (short tandem repeat) marker sequence known to be present on the X-chromosome.

3. The method according to claim 1, wherein the method is for detection and/or diagnosis of partial or complete X-chromosomal aneuploidies.

4. The method according to of claim 1, wherein the method is for detection and/or diagnosis of partial or complete X-chromosomal monosomies.

5. The method according to claim 4, wherein said chromosomal monosomy is Turner's syndrome (XO).

6. The method according to of claim 1, wherein said chromosomal disorder is Turner's syndrome (XO).

7. The method according to claim 1, wherein said chromosomal disorder is Klinefelter syndrome.

8. The method according to claim 3, wherein said X-chromosomal aneuploidy is Klinefelter syndrome.

9. The method according to claim 5, wherein the at least one pair of marker sequences known to be present on the X-chromosome and on an autosomal chromosome is the BRAF-gene on chromosome 7 and the BRAF2-gene on chromosome X.

10. The method according to claim 9, wherein the marker sequences are amplified using at least two primers selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17.

11. The method according to claim 9, wherein the marker sequences are amplified using a primer pair selected from the group consisting of SEQ ID NOs 1 and 2; SEQ ID NOs 1 and 3; SEQ ID NOs 1 and 4; SEQ ID NOs 1 and 5; SEQ ID NOs 1 and 9; SEQ ID NOs 1 and 11; SEQ ID NOs 1 and 14; SEQ ID NOs 1 and 15; SEQ ID NOs 1 and 17; SEQ ID NOs 6 and 7; SEQ ID NOs 6 and 8; SEQ ID NOs 6 and 14; SEQ ID NOs 10 and 7; SEQ ID NOs 12 and 7; SEQ ID NOs 13 and 3; SEQ ID NOs 13 and 11; SEQ ID NOs 16 and 3; and SEQ ID NOs 16 and 7.

12. The method according to claim 9, wherein the marker sequences are amplified using the primer pair SEQ ID NO. 1 and SEQ ID NO. 2.

13. The method according to claim 1, wherein the sex chromosome marker sequence(s) is/are selected from the group consisting of the amelogenin gene marker sequences (AMELX and AMELY) and the SRY gene marker sequence (on the Y chromosome).

14. The method according to claim 2, wherein the STR marker sequence is selected from the group consisting of DXS1187, DXS981 and XHPRT.

15. The method according to claim 2, wherein the at least three genetic markers comprises the following marker sequences: the BRAF-gene on chromosome 7 and the BRAF2-gene on chromosome X, the amelogenin gene marker sequences (AMELX and AMELY), and the DXS1187 STR marker sequence.

16. The method according to claim 15 wherein also at least one of the genetic markers DXS981, XHPRT and SRY is chosen.

17. The method according to claim 15 wherein also the genetic marker sequences of DXS981, XHPRT and SRY are chosen.

18. A diagnostic kit including reagents and instructions for performing the method according to claim 1.

19. A screening method, characterized in that the method according to claim 1 is used.

20. A diagnostic kit for the performing the method according to claim 1, the kit comprising a primer comprising a sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17.


SEQUENCE LISTING (17 sequences)

SEQ ID NO: 1 - DNA, 20 bp, Artificial Sequence
  Forward Primer
ggggaacggaactgattttt

SEQ ID NO: 2 - DNA, 21 bp, Artificial Sequence
  Reverse Primer
tgttgggcaggaagactctaa

SEQ ID NO: 3 - DNA, 21 bp, Artificial Sequence
  Reverse Primer
ttgttgggcaggaagactcta

SEQ ID NO: 4 - DNA, 20 bp, Artificial Sequence
  Reverse Primer
tgttgggcaggaagactcta

SEQ ID NO: 5 - DNA, 19 bp, Artificial Sequence
  Reverse Primer
gtggtgacttggggttgct

SEQ ID NO: 6 - DNA, 19 bp, Artificial Sequence
  Forward Primer
ctggggaacggaactgatt

SEQ ID NO: 7 - DNA, 21 bp, Artificial Sequence
  Reverse Primer
tgttgggcaggaagactctaa

SEQ ID NO: 8 - DNA, 21 bp, Artificial Sequence
  Reverse Primer
ttgttgggcaggaagactcta

SEQ ID NO: 9 - DNA, 18 bp, Artificial Sequence
  Reverse Primer
tggtgacttggggttgct

SEQ ID NO: 10 - DNA, 20 bp, Artificial Sequence
  Forward Primer
ctggggaacggaactgattt

SEQ ID NO: 11 - DNA, 19 bp, Artificial Sequence
  Reverse Primer
ttgttgggcaggaagactc

SEQ ID NO: 12 - DNA, 19 bp, Artificial Sequence
  Forward Primer
tggggaacggaactgattt

SEQ ID NO: 13 - DNA, 20 bp, Artificial Sequence
  Forward Primer
aaccccaagtcaccacaaaa

SEQ ID NO: 14 - DNA, 19 bp, Artificial Sequence
  Reverse Primer
ttgtggtgacttggggttg

SEQ ID NO: 15 - DNA, 20 bp, Artificial Sequence
  Reverse Primer
tttgtggtgacttggggttg

SEQ ID NO: 16 - DNA, 19 bp, Artificial Sequence
  Forward Primer
caaccccaagtcaccacaa

SEQ ID NO: 17 - DNA, 18 bp, Artificial Sequence
  Reverse Primer
gtggtgacttggggttgc